(19) United States
(12) Patent Application Publication
(10) Pub. No.: US 2020/0149117 A1
(43) Pub. Date: May 14, 2020

(54) KIT AND METHOD FOR DETECTING TARGET-TUMOR SERUM APTAMER COMPLEX

(71) Applicant: Shiqi LIAO, LANZHOU (CN)

(72) Inventors: Shiqi LIAO, LANZHOU (CN); Hongxia YUAN, LANZHOU (CN); Jiayu ZENG, LANZHOU (CN); Yi LI, LANZHOU (CN); Zhengyu LIAO, LANZHOU (CN)

(21) Appl. No.: 16/658,096

(22) Filed: Oct. 20, 2019

Related U.S. Application Data
(63) Continuation of application No. PCT/CN2018/114555, filed on Nov. 8, 2018.

(51) Int. Cl. C12Q 1/6886; C12N 15/10

(52) U.S. Cl. CPC C12Q 1/6886; C12Q 2600/158; C12N 15/1048

(57) ABSTRACT

A method and a kit for detecting a target-tumor serum aptamer complex. The method relates to the detection of a marker target in a mixed sample, where a target-specific aptamer group, is bound with the target to convert the target marker signal into an aptamer signal, which can be dynamically and quantitatively detected by multiplex real-time quantitative PCR. The kit includes magnetic beads, a blocking buffer, a detection reagent, a detergent and a real-time quantitative PCR system. The magnetic beads have a particle size of 5-5000 nm. The blocking buffer is a solution for blocking proteins. The detection reagent contains a tumor serum-specific aptamer group and a non-tumor serum-specific aptamer. The real-time quantitative PCR system comprises a primer and fluorescent probes for aptamers. This method has characteristics of rapid detection, high sensitivity, strong specificity and simultaneous detection using various aptamers.

### CROSS-REFERENCE TO RELATED APPLICATIONS

This application is a continuation of International Patent Application No. PCT/CN2018/114555 with a filing date of Nov. 18, 2018, designating the United States, now pending. The content of the aforementioned applications, including any intervening amendments thereto, are incorporated herein by reference.

### TECHNICAL FIELD

This application relates to biomedical detection, and more particularly to a method and a kit for detecting a target-tumor serum aptamer complex.

### BACKGROUND OF THE PRESENT INVENTION

With the development of SELEX technique, aptamers, as a class of novel ligands with high specificity, wide application and easy modification, have been widely used in diagnosis and treatment of diseases, screening and application of drugs, supervision of food and environment safety and biological detection.

Nucleic acid beacon ligand is a novel detecting molecule derived from the aptamer, where the nucleic acid beacon ligand is prepared by ligating a double-stranded nucleotide sequence bearing a fluorescence-labeled probe and an aptamer of a known specific target molecule to the 5′ end of the original aptamer. Therefore, the nucleic acid beacon ligand not only has the recognition capacity of the common aptamer but also plays an important role in the signal storage, transduction and amplification. Moreover, the beacon aptamer further has the characteristics of easy construction, strong specificity, high sensitivity and wide applicability. However, due to the introduction of a beacon sequence, the nucleic acid sequence of the aptamer is altered, thereby affecting the spatial structure and the affinity between the aptamer and its ligand. Therefore, if an aptamer is not changed in the structure by the treatment and the treated aptamer can also be detected by real-time quantitative PCR, the aptamer will be more applicable.

Magnetic bead, as a novel multi-functional material, is considered to be, an ideal carrier in the detection of aptamers due to its desirable biocompatibility and the presence of surface functional groups. Moreover, magnetic beads have also been widely used in the fields of food, medicine, environment and biological separation.

Serum is the most easily available sample in clinical practice and can provide a large amount of information about body function. Almost all the cells in the body are directly or indirectly in contact with the blood, so any disease may affect the serum protein to a certain extent, resulting in changes in some characteristics.

Proteins are generally detected by enzyme-linked immunosorbent assay (ELISA). Specifically, a capture antibody is first bound with an antigen in the serum; then a detecting antibody linked with a conjugating enzyme is added to form a capture antibody-antigen-detecting antibody “sandwich” complex; and finally, activity of the conjugating enzyme is measured to obtain the detection results. However, the detection range of this method is limited by the Kd value of the reaction of the capture antibody and the antigen, moreover, this method also has a low sensitivity.

There are currently two methods for detecting the nucleic acid beacon ligand. One of the methods refers to nucleic acid beacon ligand-mediated immuno-PCR detection, where the process is, similar to the ELISA and the difference is that this method uses a complex formed by a specific nucleic acid beacon ligand and a capture antibody instead of an enzyme-labeled secondary antibody corresponding to the capture antibody to target the antigen and then the detection is completed by real-time quantitative PCR. While the other method relates to nucleic acid beacon ligand-mediated PCR detection, where a nucleic acid beacon ligand corresponding to the target molecule is used as a detection molecule to bind to the target molecule, and then the bound product is eluted and separated for the real-time quantitative PCR detection. In both of the two methods, the target molecule is firstly specifically recognized by the nucleic acid beacon ligand, and the produced signal is then transmitted and finally amplified by real-time quantitative PCR to complete the detection of the target molecule. The method involving the use of a nucleic acid beacon ligand to detect a target molecule has advantages of rapid detection, high sensitivity and strong specificity.

Aptamer-mediated real-time quantitative PCR is an improved nucleic acid beacon ligand detection technique. In the nucleic acid beacon ligand, a double-stranded beacon sequence is connected to two ends of the aptamer sequence to transmit the signal to the beacon sequence through the aptamer after the aptamer binds to the target molecule, and the beacon is then detected by real-time quantitative PCR, directly achieving the detection for the target molecule. However, in the practical process, the effect of the aptamer on the target molecule often cannot be achieved through the single action of a single strand, but is often completed by the synergistic action of several strands. In addition, the beacon double strand also affects the spatial structure of the aptamer, which consequently affects the aptamer structure and the synergistic action among the aptamers, thereby affecting the binding between the aptamer and the target molecule and the detection results. Therefore, there is a need to improve the detection method to overcome the defects in the prior art.

### SUMMARY OF PRESENT INVENTION

This application provides a method and a kit for detecting a target-tumor serum aptamer complex to overcome the above defects in the prior art.

The technical solutions of this application are described as follows.

This application discloses a kit for detecting a target-tumor serum aptamer complex, comprising: magnetic beads, a blocking buffer, a detection reagent, a detergent and a real-time quantitative PCR system; wherein:

the magnetic beads have a particle size of 5-5000 nm;

the blocking buffer is a solution for blocking proteins;

the detection reagent comprises a tumor serum-specific aptamer group and a non-tumor serum-specific aptamer; and

the real-time quantitative PCR system comprises a primer and fluorescent probes for aptamers.

In an embodiment, the magnetic beads are selected from a 0.01 M binding buffer (5 mL, pH 7.4) containing 50% of mapetic beads with a particle size of 5-5,000 nm; the detection reagent comprises a tumor serum-specific aptamer group of gastric cancer (G-seq), liver cancer (H-seq) and lung cancer (L-seq) and a non-tumor (N-seq) serum-specific aptamer, wherein individual aptamers in the detection reagent is a mixture of 109 molecular copies and 0.1 binding buffer; the detergent comprises a first detergent and a second detergent, wherein the first detergent is selected from a 0.01 M binding, buffer (10 mL, pH 7.4) containing 0.17% Tween, and the second detergent is selected from a 3×SSC (10 mL) containing citric acid and sodium chloride; and the real-time quantitative PCR system is selected from a PCR system comprising a pair of primers and a plurality of fluorescent probes with different emission wavelengths for aptamers.

In an embodiment, the tumor serum-specific aptamer group and the non-tumor serum-specific aptamer are both obtained by a two-way thermal cycle subtractive SELEX. The tumor serum-specific aptamer group is selected from a gastric cancer, liver cancer or lung cancer serum-specific aptamer group screened by two-way (or multi-way) subtractive SELEX for anon-tumor serum (a mixture of 10 or more serum samples), and the non-tumor serum-specific aptamer is correspondingly obtained by two-way (or multi-way) subtractive SELEX for a gastric cancer serum, a liver cancer serum or a lung cancer serum (respectively a mixture of 10 or more serum samples), wherein the gastric cancer serum, the liver cancer serum and the lung cancer serum are mutually subtractive targets with respect to the non-tumor serum, respectively. Respective serum-specific aptamers in the detection reagent preferably have a molecular copy number of 106-109.

In an embodiment, aptamers in the tumor serum-specific aptamer group and the non-tumor serum-specific aptamer respectively correspond to the fluorescent probes, that is, the fluorescent probes are designed according to the sequence of respective aptamers. Specifically, the fluorescent probes comprise at least one of an MGB probe, a TaqMan probe and a molecular beacon, and have a sequence of 5-25 bp, wherein 3′ and 5′ ends of the sequence of the probe are respectively provided with a fluorescent group including FAM, HEX and TET and a quencher including TAMRA and BHQ to assist the real-time quantitative detection.

In an embodiment, a surface of the capture magnetic bead is provided with a functional group or a capture molecule capable of coupling with a target molecule, the functional group comprises at least one of an epoxy group, a carboxyl group, an amino group and NHS, and is capable of chemically coupling with the target molecule, and the capture molecule is one or more of an antigen, an antibody, an affinity protein and an aptamer, and is capable of capturing the target molecule by immune-binding or binding between a protein ligand and an aptamer; and the target molecule comprises at least one of nucleic acid, protein, lipid and amino acid.

In an embodiment, the primer is a primer of an aptamer, and a probe for the primer has a sequence consisting of 5-25 consecutive bases on a sequence; and 3′ and 5′ ends of the sequence of the probe are respectively provided with a quencher and a fluorescent group.

In an embodiment, the blocking buffer comprises skim milk powder and casein, or bovine serum albumin.

This application also provides a method of using the kit to detect the target-tumor serum aptamer complex, comprising the following steps:

1) preparation of a sample to be detected

removing blood cells and blood lipids in a blood sample by separation to obtain a serum, i.e., the sample to be detected;

2) capturing of a target molecule

mixing the magnetic beads with the sample to be detected followed by incubation at 37° C. for 1 h to produce a magnetic bead-target molecule complex, wherein for non-specific binding, the complex is further required to be blocked with the blocking buffer at 37° C.′ for 1 h; washing the magnetic bead-target molecule complex three times with the first detergent and each, for 3 min followed by magnetic separation to collect magnetic beads;

3) binding of a beacon ligand

heating the detection reagent at 95° C. for 5 min; rapidly cooling the detection reagent in a ice water bath for 5 min; adding the detection reagent to the magnetic beads for binding at 37° C. for 1 h followed by magnetic separation to remove a supernatant;

4) washing

washing the magnetic beads once with 0.5 mL of the second detergent for 3 min followed by magnetic separation; and washing the magnetic beads three times with 0.5 mL of the first detergent and each for 3 min followed by magnetic separation to collect the magnetic beads;

5) extraction of the ligand

adding 15 μL of 1 PCR buffer to the magnetic beads followed by heating at 95° C. for 5 min and magnetic separation to collect a supernatant; and

6) detection of the ligand

transferring 2 μL of the supernatant obtained in step (5) to 18 μL of the real-time quantitative PCR system followed by PCR detection to collect and analyze the data, or by genetic sequencing to complete the qualification and quantification of multi-target.

The step (1) specifically comprises the following steps: collecting the blood sample by venipuncture to a test tube containing an anticoagulant; immediately shaking the test tube gently to mix the blood sample and the anticoagulant uniformly; centrifuging the reaction mixture at 3,000 rpm for 10 min to collect a supernatant; and storing the supernatant at −80° C. for 30 min followed by centrifugation at 12,000 g for 30 min to remove the blood lipids and obtain the serum to be detected; where the serum to be detected can be further treated sequentially by mixing with water and acetonitrile in a ratio of 1:2:0.5, low-temperature centrifugation at 5000 rpm for 30 min and collection of a supernatant to remove high-abundance proteins.

In an embodiment, the step (6) comprises the step of: according to the actual needs, detecting the ligand or ligand group specifically binding to the magnetic beads by multiple real-time quantitative PCR detection, multi-library screening and multi-primer detection, genetic sequencing or other methods. The qualification and quantification of a specific marker group can be achieved through the detection of the aptamer group by multiple real-time quantitative PCR (or genetic sequencing).

It is not required to connect any sequence to the aptamer in the real-time quantitative PCR detection of aptamers, so that the spatial structure of the aptamer is not changed. The probe for the real-time quantitative PCR detection has a sequence consisting of 5-25 consecutive bases on a sequence of the aptamer. Therefore, the structure of the aptamer and the binding between the aptamer and the ligand are not required to be modified and the detection is improved with respect to sensitivity.

This application has the following beneficial effects.

1. The kit of this application is used to detect a complex target, where the specific aptamer group binds with a serum-specific target to convert the protein signal of the target-serum marker complex into a nucleic acid signal, which can be dynamically and quantitatively detected by real-time quantitative PCR. This detection method can convert signals of multiple target molecules into nucleic acid signals through the aptamer, having the characteristics of rapid detection, high sensitivity, strong specificity and simultaneous detection of various ligands.

2. This application uses magnetic beads as, a carrier to bind the target molecule, and involves the magnetic separation of the detection molecule, allowing fora simple operation.

3. The aptamer in this application is a non-tumor (N-seq), gastric cancer (G-seq), liver cancer (H-seq) or lung cancer (L-seq) serum-specific aptamer sequence obtained by a two-way thermal cycle subtractive SELEX. The fluorescent probes are respectively designed according to the aptamers, and the target molecule signal can be exponentially amplified by PCR amplification.

4. A non-tumor (N-seq) serum aptamer can specifically recognize the marker in the non-tumor serum, suitable as a negative control in the, tumor serum detection.

5. The detection reagent used herein consists of a non-tumor (N-seq) serum aptamer and gastric cancer (G-seq), liver cancer (H-seq) and lung cancer (L-seq) serum-specific aptamers, which can relatively clearly determine whether there is a tumor marker or a non-tumor marker in the serum through the detection.

### DESCRIPTION OF THE DRAWINGS

This application will be described below in detail with reference to the drawings and embodiments.

FIG. 1 schematically shows the principle of detecting a tumor and non-tumor serum by multiplex real-time quantitative PCR according to Example 2 of the present invention.

### DETAILED DESCRIPTION OF PREFERRED EMBODIMENTS

This application will be further illustrated with reference to the embodiments.

The kit used in the following examples exemplarily includes the following reagents:

reagent 1: magnetic beads prepared by dissolving 50% of capture magnetic beads with a particle size of 5-5,000 nm in 5 mL of 0.01 M binding buffer (pH 7.4);

reagent 2: a detection reagent of gastric cancer (G-seq), liver cancer (H-seq) and lung cancer (L-seq) tumor serum-specific aptamers and a detection reagent of a non-tumor (N-seq) serum-specific aptamer, prepared by mixing respective aptamers in 0.1× binding buffer respectively at a molecular copy number of 109;

reagent 3: a blocking buffer (10 mL) containing skim milk powder and casein;

reagent 4: a first detergent (10 mL) prepared by mixing 0.17% Tween in a 0.01 M binding buffer (pH 7.4);

reagent 5: a second detergent (10 mL) prepared by dissolving citric acid and sodium chloride in 3×SSC; and

reagent 6: a real-time quantitative PCR system (1 mL) referring to a PCR system containing a pair of primers and aptamer fluorescent probes with different emission wavelengths.

### EXAMPLE 1

### Real-Time Quantitative PCR Detection of Tumor Serum and Non-Tumor Serum

Steps of this example were described as follows.

### 1) Preparation of a Sample to be Detected

A blood sample was collected by venipuncture to a test tube containing an anticoagulant. The test tube was immediately shaken gently to mix the blood and the anticoagulant uniformly and centrifuged at 3000 rpm for 10 min to collect a supernatant. The supernatant was stored at −80° C. for 30 min and centrifuged at 12,000 g for 30 min to remove blood lipids and obtain a serum. Then the serum was mixed with water and acetonitrile in a ratio of 1:2:0.5 and centrifuged at a low temperature and 5,000 rpm for 30 min to remove the high-abundance proteins and the obtained supernatant was the serum to be detected.

### 2) Capturing of a target molecule by magnetic beads

Two parts of NHS-based agar magnetic beads were respectively added at an equal volume of 50 μL, to two 1.5 mL EP tubes, which were labeled as H1 and H2, respectively. The H1 and H2 magnetic beads were respectively added with 50 μL of the serum to be detected, incubated at 37° C. for 1 h, blocked with a protein blocking buffer at 37° C. for 1 h, washed with the first detergent three times and each for 3 min and magnetically separated to collect the magnetic beads.

### 3) Binding of a Ligand

200 μL of the mixed detection reagent of gastric cancer (G-seq), liver cancer (H-seq) and lung cancer (L-seq) aptamers and 200 μL of the detection reagent of a non-tumor (N-seq) aptamer were heated at 95° C. for 5 min, immediately cooled in an ice water bath for 5 min, and then respectively added to the H2 and H1 tubes for binding at 37° C. for 1 h. The reaction mixtures in the two tubes were respectively magnetically separated and the obtained supernatants were both discarded.

### 4) Washing

The two tubes were respectively washed with 0.5 mL of the second detergent three times and each for 3 min, and then washed with 0.5 mL of the first detergent three times and each for 3 min.

### 5) Preparation of a Detection Template

The H1 and H2 magnetic beads were respectively added with 15 of 1×PCR buffer, heated at 95° C. for 5 min and magnetically separated to collect the supernatants.

### 6) Detection

2 μL of the supernatants obtained in step (5) in the H1 and H2 tubes were respectively added to 18 μL of a real-time quantitative PCR reaction system (SYBRGreen I) for real-time quantitative PCR detection. The data were collected and processed.

The detection results were analyzed as follows: (1) CT value of the H1 supernatant was less than that of the H2 supernatant, which indicated that the serum sample was a non-tumor serum; and (2) CT value of the H1 supernatant was greater than that of the H2 supernatant, which indicated that the serum sample was a tumor serum.

### EXAMPLE 2

### Multiplex Real-Time Quantitative PCR Detection of Tumor and Non-Tumor Serum

Steps of this example were described as follows.

### 1) Preparation of a Sample to be Detected

A blood sample was collected by venipuncture to a test tube containing an anticoagulant. The test tube was immediately shaken gently to mix the blood and the anticoagulant uniformly and centrifuged at 3,000 rpm for 10 min to collect a supernatant. The supernatant was stored at −80° C. for 30 min and centrifuged at 12,000 g for 30 min to remove blood lipids and obtain a serum. Then the serum was mixed with water and acetonitrile in a ratio of 1:2:0.5 and centrifuged at a low temperature and 5,000 rpm for 30 min to remove the high-abundance proteins and the obtained supernatant was the serum to be detected.

### (2) Preparation of a Magnetic Bead-Target Molecule Complex

50 μL of 1 part of capture agar magnetic beads was added to a 1.5 mL EP tube, added with 50 μL of the serum to be detected, incubated at 37° C. for 1 h, washed with the first detergent three times and each for 3 min, and magnetically separated to collect the magnetic beads. It should be noted that the aptamer capture agar magnetic beads were prepared as follows: magnetic beads were coupled with streptavidin by chemical bonds and then bound with a biotinylated ligand through the streptavidin to form the capture magnetic beads. The ligand for capturing the target molecule and the detection ligand may be the same molecule, or target molecule-specific ligands respectively screened from different nucleic acid libraries. Other capture magnetic beads may also chemically couple with an antibody or antigen to form the capture magnetic bead of this example, or directly capture the target molecule by chemical coupling.

### (3) Binding of a Ligand

200 μL of the mixed detection reagent of gastric cancer (G-seq), liver cancer (H-seq), lung cancer (L-seq) and non-tumor (N-seq) aptamers was heated at 95° C. for 5 min, immediately cooled in an ice water bath for 5 min, and then added to the magnetic beads obtained in step (2) for binding at 37° C. for 1 h. The reaction mixture was magnetically separated and the obtained supernatant was discarded.

### (4) Washing

The magnetic beads were washed with 0.5 mL of the second detergent three times and each for 3 min, and then washed with 0.5 mL of the first detergent three times and each for 3 min.

### (5) Preparation of a Detection Template

The magnetic beads were added with 15 μL of 1×PCR buffer, heated at 95° C. for 5 min and magnetically separated to collect a supernatant.

### (6) Multiplex PCR Detection

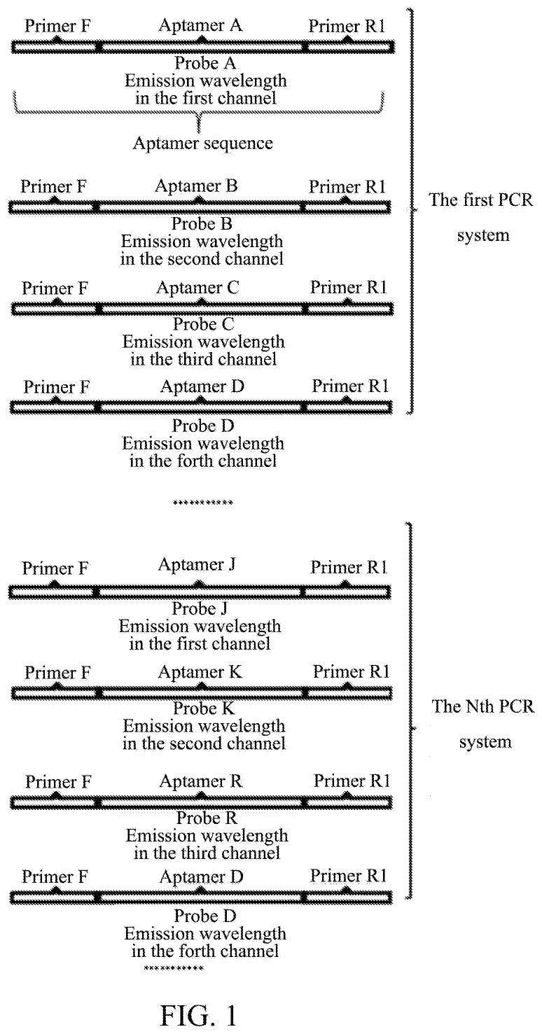

2 μL of the supernatant obtained in step (5) was added to 18 μL of a real-time quantitative PCR system and detected according to the principle shown in FIG. 1. The PCR system contained upstream and downstream primers P7 and P11 of the aptamer and four TaqMan probes of different wavelengths respectively for the labeling of non-tumor (N-seq), gastric cancer (G-seq), liver cancer (H-seq) and lung cancer (L-seq) aptamers, where the emission wavelengths of the four probes were respectively in accordance with the detection wavelengths of the four channels for the multiplex real-time quantitative PCR. The data were collected and processed accordingly.

The detection results were analyzed as follows: (1) the CT value of the first channel was lower than those of the second, third and forth channels, indicating that the serum sample referred to a non-tumor serum; (2) the CT value of the second channel was lower than those of the first, third and forth channels, indicating that the serum sample may refer to a gastric cancer serum; (3) the CT value of the third channel was lower than those of the first, second and forth channels, indicating that the serum sample may refer to a liver cancer serum; and (4) the CT value of the forth channel was lower than those of the first, second and third channels, indicating that the serum sample may refer to a lung cancer serum.

The following details should be noted.

1. When the number of samples was greater than that of the channels of the real-time quantitative PCR detection, that was, there were more than 4 samples to be detected, a control can be introduced to each PCR detection, that was, one of the four channels for the control and the rest three for the samples to be detected. For example, if there were 10 types of tumors to be detected, they can be divided into four groups for PCR detection and in each group, a TaqMan probe carrying the non-tumor aptamer was used as the control to eliminate the errors among groups.

2. If there were at least 2 types of aptamers for each tumor, these aptamers can be detected in one PCR system in the use of TaqMan probes of the same emission wavelength.

3. In this example, appropriate nucleic acid libraries can be selected according to the sample marker to screen aptamers. In this way, PCR systems containing different primers can be used to detect the same detection template. acquiring information about the marker and determining the tumor type of the sample.

### EXAMPLE 3

### Genetic Sequencing of Tumor and Non-Tumor Serums

This example included the following steps.

### (1) Preparation of a Sample to be Detected

A blood sample was collected by venipuncture to a test tube containing an anticoagulant. The test tube was immediately shaken gently to mix the blood and the anticoagulant uniformly and centrifuged at 3,000 rpm for 10 min to collect a supernatant. The supernatant was stored at −80° C. for 30 min and then centrifuged at 12,000 g for 30 min to remove blood lipids and obtain a serum. Then the serum was mixed with water and acetonitrile in a ratio of 1:2:0.5 and centrifuged at a low temperature and 5,000 rpm for 30 min to remove the high-abundance proteins and the obtained supernatant was the serum to be detected.

### (2) Preparation of Magnetic Bead-Target Molecule Complex

50 μL of 1 part of capture agar magnetic beads was added to a 1.5 mL EP tube, added with 50 μL of the serum to be detected, incubated at 37° C. for 1 h, washed with the first detergent three times and each for 3 min, and magnetically separated to collect the magnetic beads. It should be noted that the aptamer capture agar magnetic beads were prepared as follows: magnetic beads were coupled with streptavidin by chemical bonds and then bound with a biotinylated ligand through the streptavidin to form the capture magnetic beads. The ligand for capturing the target molecule and the detection ligand may be the same molecule, or target molecule-specific ligands respectively screened from different nucleic acid libraries. Other capture magnetic beads may also chemically couple with an antibody or antigen to form the capture magnetic bead of this example, or directly capture the target molecule by chemical coupling.

### (3) Binding of a Ligand

200 μL of the mixed detection reagent of gastric cancer (G-seq), liver cancer (H-seq), lung cancer (L-seq) and non-tumor (N-seq) aptamers respectively with 109 copies was prepared, heated, at 95° C. for 5 min, immediately cooled in an ice water bath for 5 min, and then added to the magnetic beads obtained, in step (2) for binding at 37° C. for 1 h. The reaction mixture was, magnetically separated and the obtained supernatant was discarded.

### (4) Washing

The magnetic beads were washed with 0.5 mL of the second detergent three times and each for 3 min, and then washed with 0.5 mL of the first detergent three times and each for 3 min.

### (5) Preparation of a Detection Template

The magnetic beads were added with 15 μL of 1 PCR buffer, heated at 95° C. for 5 min and magnetically separated to collect a supernatant.

### (6) Genetic Sequencing

The supernatant obtained in step (5) was detected by second- or third generation genetic sequencing, and then each aptamer was analyzed. The copy number of the aptamer was associated with the number of the specific target, indicating the type of the serum.

It should be understood that those skilled in the art can make some modifications or changes to this application based on the above description, and these modifications or changes should all fall within the scope of the appended claims of the invention.

These embodiments are merely illustrative of the invention and are not intended to limit the invention. Any modifications made without departing from the spirit of the invention should fall within the scope of the invention.

We claim:

1. A kit for detecting a target-tumor serum aptamer complex, comprising:
magnetic beads, a blocking buffer, a detection reagent, a detergent and a real-time quantitative PCR system;
wherein:
the magnetic beads have a particle size of 5-5000 nm;
the blocking buffer is a solution for blocking proteins;
the detection reagent comprises a tumor serum-specific aptamer group and a non-tumor serum-specific aptamer; and
the real-time quantitative PCR system comprises a primer and fluorescent probes for aptamers.

2. The kit according to claim 1, characterized in that the tumor serum-specific aptamer group and the non-tumor serum-specific aptamer are both obtained by a two-way thermal cycle subtractive SELEX.

3. The kit according to claim 1, characterized in that aptamers in the tumor serum-specific aptarner group and the non-tumor serum-specific aptamer respectively correspond to the fluorescent probes.

4. The kit according to claim 3, characterized in that the fluorescent probes comprise at least one of an MGB probe, a TaqMan probe and a molecular beacon, and are designed according to respective aptarner sequences.

5. The kit according to claim 1, characterized in that, a surface of the capture mapetic bead is provided with a functional group or a capture molecule capable of coupling with a target molecule; the functional group comprises at least, one of an epoxy group, a carboxyl group, an amino group and NHS, and, is capable of coupling with the target molecule by a chemical group; the capture molecule is one or more of an antigen, an antibody, an affinity protein and an aptamer, and is capable of capturing the target molecule by immuno-binding to a protein ligand or an aptamer; and the target molecule comprises at least one of nucleic acid, protein, lipid and amino acid.

6. The kit according to claim 1, characterized in that the detection reagent contains gastric cancer, liver cancer and lung cancer serum-specific aptamers screened by subtractive SELEX for non-tumor serum and a non-tumor serum-specific aptamer screened by subtractive SELEX for tumor serum.

7. The kit according to claim 1, characterized in that the detergent comprises a first detergent and, a second detergent; wherein the first detergent is a binding buffer containing Tween and the second detergent is SSC containing citric acid and sodium chloride.

8. The kit according to claim 1, characterized in that the blocking buffer comprises skim milk powder and casein, or bovine serum albumin.

9. The kit according to claim 1, characterized in that the primer is a primer of an aptamer, and a probe for the primer has a sequence consisting of 5-25 consecutive bases on a sequence of the aptamer; and 3′ and 5′ ends of the sequence of the probe are respectively provided with a quencher and a fluorescent group.
